(19) United States
(12) Patent Application Publication
(10) Pub. No.: US 2023/0309986 A1
(43) Pub. Date: Oct. 5, 2023

(54) TISSUE ANCHORS FOR ENDOSCOPICALLY CLOSING GASTROINTESTINAL DEFECTS

(71) Applicant: Covidien LP, Mansfield, MA (US)

(72) Inventor: Ryan W. Bertwell, San Jose, CA (US)

(21) Appl. No.: 18/100,689

(22) Filed: Jan. 24, 2023

Related U.S. Application Data
(60) Provisional application No. 63/324,690, filed on Mar. 29, 2022.

(51) Int. Cl. A61B 17/04; A61B 17/064

(52) U.S. Cl. CPC A61B 17/0401; A61B 17/064; A61B 2017/0409

(57) ABSTRACT

A tissue anchor for use in repairing a wound after an endoscopic resection procedure includes a hollow cylindrical body configured to penetrate tissue during a distal insertion of the tissue anchor into the tissue, and a plurality of tines coupled to the cylindrical body. The tines are configured to transition between a stowed configuration in which the tissue anchor is configured to pass through a working channel of an endoscope, and a deployed configuration, in which the tines project outwardly relative to the cylindrical body to secure the tissue anchor in the tissue.

### CROSS-REFERENCE TO RELATED APPLICATIONS

This application claims the benefit of the filing date of provisional U.S. Patent Application No. 63/324,690 filed on Mar. 29, 2022.

### FIELD

The present technology is related generally to tissue anchors for endoscopically closing a wound.

### BACKGROUND

Endoscopic resection, such as endoscopic submucosal dissection (ESD), endoscopic mucosal resection (EMR), peroral endoscopic myotomy (POEM), and full thickness resection, has been accepted as a first choice of the treatment for early stage GI carcinomas because of less invasiveness and lower cost. ESD, for example, allows for an en bloc resection and accurate histopathological diagnosis regardless of the size and location of a lesion or an existence of severe fibrosis at the submucosal layer.

After endoscopic resection, such as ESD, the submucosa or muscle layer may be exposed. For example, after an ESD, lesions in the mucosal and submucosal space are removed leaving an exposed area in the GI tract. Oftentimes, clinicians decide not to close the opening in the mucosal layer. However, there has been recent interest and evidence that closing the mucosal layer may lead to reduced complications.

### SUMMARY

In accordance with an aspect of the disclosure, a tissue anchor used in repairing a wound after an endoscopic resection procedure is provided. The tissue anchor includes an elongated main body and two or more tines coupled to the main body. The main body defines a channel therethrough and has a proximal end portion, and a distal end portion configured to puncture tissue. The tines are configured to move relative to the main body between a stowed configuration, and a deployed configuration, in which the tines flare outwardly relative to the main body.

In aspects, the tines may be linear in the stowed configuration.

In aspects, the tines may be arcuate in the deployed configuration.

In aspects, the tines may be fabricated from a shape memory material configured to transition the tines from the stowed configuration to the deployed configuration when the tines reach a preset, threshold temperature.

In aspects, the tines may have an outer surface that is circumferentially-aligned with or positioned radially inward from an outer surface of the main body when the tines are in the stowed configuration.

In aspects, each of the tines may have a first end attached to the distal end portion of the main body, and a free second end.

In aspects, the free second end of each of the tines may be perpendicular to a longitudinal axis defined by the main body when the at least two tines are in the deployed configuration.

In aspects, the tines may be positioned on the main body in opposition to one another.

In aspects, the tines may be four tines positioned circumferentially spaced from one another about the main body.

In aspects, the main body may define at least two elongated cutouts therein. The tines may be positioned within the respective elongated cutouts when the tines are in the stowed configuration.

In aspects, the main body may have a tubular shape, and the channel may be cylindrical.

In aspects, the channel may be opened at each of the proximal and distal end portions of the main body.

In aspects, the distal end portion of the main body may have at least one tooth extending distally therefrom. The at least one tooth may be configured to puncture the tissue during a distal insertion of the main body into the tissue.

In aspects, the distal end portion of the main body may have a distal-most edge having a first portion having a concave shape, and an opposite second portion having a concave shape. The at least one tooth may be two teeth that extend distally from the respective first and second portions of the distal-most edge.

In aspects, when the tines are in the deployed configuration, the tines may have a convex, proximal-facing surface and a concave, distal-facing surface.

In aspects, the tines and the main body may be monolithically formed with one another.

In accordance with another aspect of the disclosure, a tissue anchor used in repairing a wound after an endoscopic resection procedure is provided. The tissue anchor includes an elongated main body, at least one tooth, and at least two tines. The main body includes a proximal end portion, and a distal end portion. The main body defines a channel that extends from the proximal end portion to the distal end portion. The at least one tooth extends distally from the distal end portion of the main body and are configured to puncture tissue during a distal insertion of the main body into the tissue. The tines have a first end coupled to the main body, and a free second end. The tines are configured to transition between a stowed configuration, and a deployed configuration. The free second end of the tines are positioned a greater radial distance from an outer surface of the main body when the tines are in the deployed configuration than when the tines are in the stowed configuration.

In aspects, the tines may be linear in the stowed configuration, and arcuate in the deployed configuration.

In aspects, the tissue anchor may be formed from a superelastic (e.g., nickel titanium) hypotube.

In aspects, the tines may be resiliently biased toward the deployed configuration.

In aspects, the tissue anchor may have a first overall diameter when the tines are in the stowed configuration, and a second overall diameter when the tines are in the deployed configuration. The second overall diameter is greater than the first overall diameter.

### BRIEF DESCRIPTION OF THE DRAWINGS

The accompanying drawings, which are incorporated in and constitute a part of the specification, illustrate embodiments of the disclosure and, together with a general description of the disclosure given above as well as the detailed description of the embodiment or embodiments given below, serve to explain the principles of this disclosure.

FIG. 1 is a side view illustrating a tissue anchor in a deployed configuration;

FIG. 2 is another side view illustrating the tissue anchor of FIG. 1 in a stowed configuration;

FIG. 3 is a top perspective view illustrating the tissue anchor of FIG. 1 in the deployed configuration;

FIG. 4 is a side view illustrating the tissue anchor of FIG. 2 in the deployed configuration and in penetrating engagement with tissue; and

FIG. 5 is a top view illustrating the tissue anchor of FIG. 1 in the deployed configuration.

### DETAILED DESCRIPTION

As used herein, the term “distal” refers to the portion that is being described which is further from a clinician, while the term “proximal” refers to the portion that is being described which is closer to a clinician. As used herein, the terms parallel and perpendicular are understood to include relative configurations that are substantially parallel and substantially perpendicular up to about +/— 10 degrees from true parallel and true perpendicular. Further, to the extent consistent, any of the aspects described herein may be used in conjunction with any or all of the other aspects described herein.

The present disclosure is generally directed to a surgical system used in closing a defect after performing an endoscopic submucosal dissection. The surgical system includes a plurality of tissue anchors configured for insertion through a working channel of an endoscope. Each of the tissue anchors includes a main body and a series of flexible tines that protrude outwardly from the main body to puncture the mucosa and lock into the submucosa. The main body may have one or more rigid teeth extending distally therefrom configured to puncture the mucosa upon distal insertion of the tissue anchor into tissue. The tissue anchor may be manufactured from a single nitinol hypotube whereby the overall shape including the rigid teeth and the locking tines may be laser cut. The tines may be heat set to protrude outwardly from the main body. Additionally, the tines may be sufficiently thin and flexible to flex into unexpanded/stowed shape when loaded in the endoscope working channel, then flare out to the heat set shape once punctured through the mucosa. Additionally, the hollow tube design of the anchors may allow for the acceptance of other parts which may include suture management features to aid in the closure of the exposed area. These and other aspects of the present disclosure are described in greater detail below.

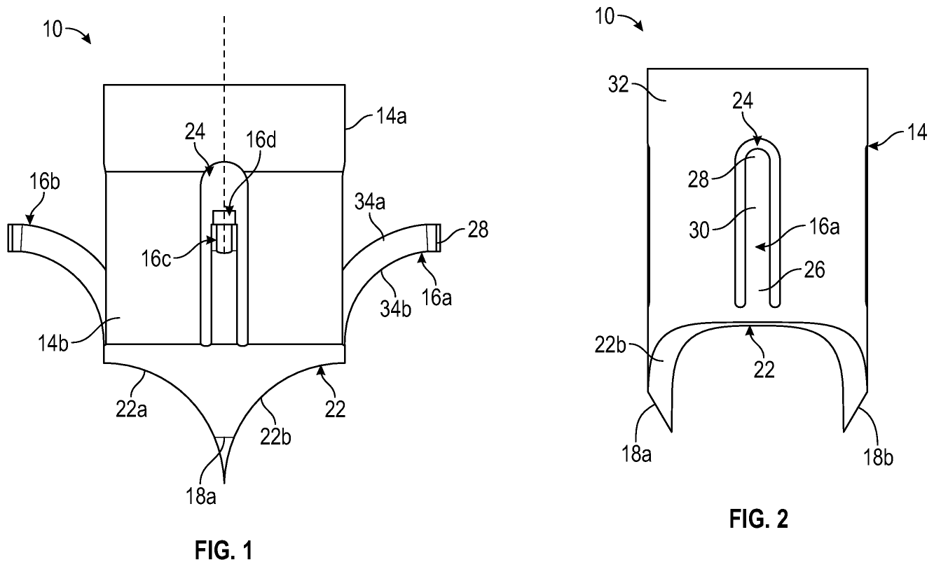

FIGS. 1-5 illustrate a tissue anchor 10 configured for anchoring into submucosal tissue. The tissue anchor 10 is configured to passed through a working channel of an endoscope (not shown) and deployed therefrom into a surgical site. It is contemplated that the present disclosure provides a surgical system including a plurality of the tissue anchors 10 configured to be positioned around a wound (e.g., a wound created by the resection of a lesion at the submucosal layer) and approximated toward one another (e.g., using a suture) to close the wound.

Each of the tissue anchors 10 includes a main body 14, two teeth or blades 18a, 18b extending distally from the main body 14, and tines 16a, 16b, 16c, 16d (or spikes) coupled to the main body 14. In aspects, the tissue anchors 10 may each include less or more than four tines 16a, 16b, 16c, 16d and less or more than two teeth 18a, 18b. The tines 16a, 16b, 16c, 16d are circumferentially spaced from one another about the main body 14. The tissue anchor 10 may be fabricated from a single tube of shape memory material (e.g., nickel titanium). In other aspects, the tissue anchor 10 may be fabricated from stainless steel or other suitable materials. In aspects, rather than being monolithically formed from a single tube, the main body 14, tines 16a, 16b, 16c, 16d, and/or teeth 18a, 18b may each be separate components that are coupled to one another using a suitable fastening technique, such as, for example, welding, brazing, soldering, adhesives, or the like.

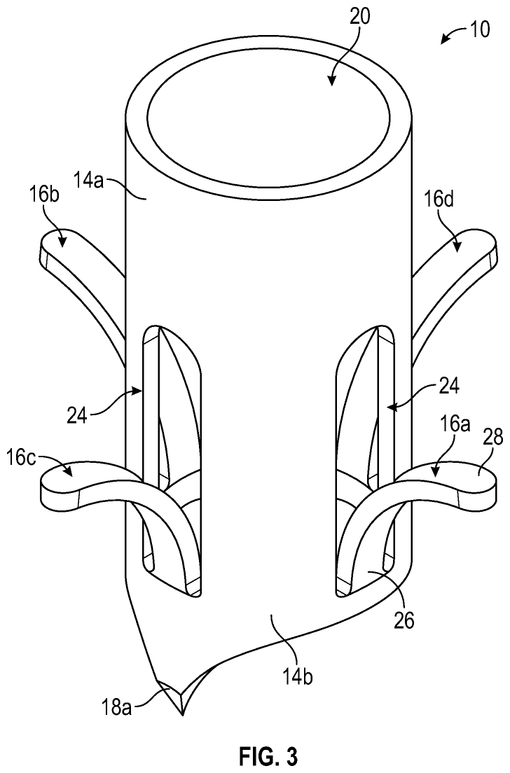

The main body 14 of the tissue anchor 10 has a tubular shape and defines a cylindrical channel 20 (FIG. 3) therethrough. In aspects, the main body 14 and channel 20 may assume other suitable shapes. The main body 14 has a proximal end portion 14a and a distal end portion 14b that are each opened such that surgical tools, sutures, or the like may be passed into and/or out of the main body 14 during a surgical procedure. The teeth 18a, 18b extend distally from the distal end portion 14b of the main body 14 or may form a distal-most part of the distal end portion 14b of the main body 14. The teeth 18a, 18b are configured to puncture tissue during a distal insertion of the main body 14 into the tissue. The distal end portion 14b of the main body 14 has a distal-most edge 22 having a first portion 22a having a concave shape, and an opposite second portion 22b having a concave shape. The first and second portions 22a, 22b of the distal-most edge 22 converge to form the first and second teeth 18a, 18b. In aspects, the distal-most edge 22 may be sharp to facilitate penetration of the tissue anchor 10 into tissue.

The main body 14 defines a plurality of elongated cutouts 24 therein. The cutouts 24 are radially spaced from one another about the main body 14 and are in communication with the channel 20 of the main body 14. The tines 16a, 16b, 16c, 16d are each received in the respective cutouts 24 of the main body 14 such that when the tines 16a, 16b 16c, 16d are in a stowed configuration (FIG. 2), the tines 16a, 16b 16c, 16d are concealed within the cutouts 24. Since each of the tines 16a, 16b 16c, 16d are identical or substantially similar, only the first tine 16a will be described in detail herein.

The first tine 16a may be formed by being cut out of the main body 14 (e.g., by laser cutting) and has a first end 26 that is fixed to the distal end portion 14b of the main body 14, and a free second end 28. The free second end 28 of the tine 16a may be rounded/atraumatic or traumatic/sharp configured to penetrate tissue. The tine 16a is fabricated from a shape memory material (e.g., nickel titanium) such that the tine 16a is resiliently biased toward an expanded or deployed configuration (FIGS. 1 and 3-5). The tine 16a may be heat set to a threshold temperature, such as, for example, a temperature above room temperature and less than or equal to a baseline body temperature (e.g., from between about 80 degrees to about 90 degrees). As such, when the tine 16a contacts tissue, the heat from the tissue raises the temperature of the tine 16a to the preset threshold temperature to automatically deploy the tine 16a. In other aspects, instead of the tine 16a being fabricated from a shape memory material, the tine 16a may be fabricated from an elastic material (e.g., a thermoplastic) that is resiliently biased to the deployed configuration.

With reference to FIG. 2, when the tissue anchors 10 are stowed within a working channel of an endoscope or a cannula (not shown), the tines 16a, 16b, 16c, 16d assume a linear shape (e.g., either due to the size of the working channel or the temperature of the tines 16a, 16b, 16c, 16d being below the preset threshold) in which an outer surface 30 of the tines 16a, 16b, 16c, 16d is circumferentially aligned with an outer surface 32 of the main body 14. In some aspects, the outer surface 30 of the tines 16a, 16b, 16c, 16d may be positioned radially inward of the outer surface 32 of the main body 14 when the tines 16a, 16b, 16c, 16d are in the stowed configuration.

With reference to FIGS. 1 and 3-5, in the deployed configuration, the tines 16a, 16b, 16c, 16d flare outwardly from the main body 14 whereby the tines 16a, 16b, 16c, 16d each have a generally proximal-facing surface 34a (FIG. 1) having a convex shape, and a generally distal-facing surface 34b having a concave shape, which together assist in securing the tines 16a, 16b in tissue. As such, the tines 16a, 16b, 16c, 16d may have an arcuate shape along their lengths when deployed. The free second end 28 of the tines 16a, 16b, 16c, 16d may extend perpendicularly relative to a longitudinal axis of the main body 14 when the tines 16a, 16b, 16c, 16d are in the deployed configuration.

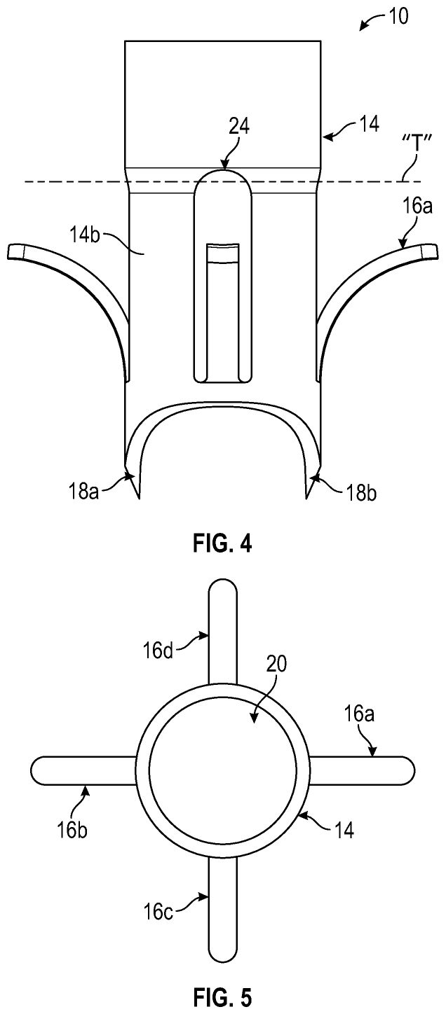

In use, during an endoscopic resection, such as ESD, lesions in the mucosal and submucosal space are removed leaving an exposed area in the GI tract. To close the exposed area of wound in the GI tract, the surgical system of the present disclosure may be utilized. More specifically, a plurality of the tissue anchors 10 may be passed through a working channel of an endoscope. Each of the tissue anchors 10 are positioned, in spaced relation from one another, around an outer periphery of the wound and translated distally to puncture the tissue with the teeth 18a, 18b of the tissue anchor 10. Distal translation of the tissue anchors 10 is continued until at least the distal end portion 14b of the main body 14 is beneath the tissue surface “T,” as shown in FIG. 4.

As the tines 16a, 16b, 16c, 16d contact the tissue, the temperature of the tines 16a, 16b, 16c, 16d increases to the preset threshold temperature and the tines 16a, 16b, 16c, 16d transition from the stowed configuration (FIG. 2) to the deployed configuration (FIG. 4). The free second ends 28 of the tines 16a, 16b, 16c, 16d project radially outward from the main body 14 and penetrate or otherwise engage the tissue to secure the tissue anchors 10 in the tissue. Due to the tines 16a, 16b, 16c, 16d being engaged with the tissue, rotation or proximal withdrawal of the tissue anchors 10 from the tissue is inhibited.

A suture or tether (not explicitly shown) may be threaded through one or more of the cutouts 24 or the channel 20 of each of the tissue anchors 10 and pulled to constrict the tissue anchors 10 about the wound, thereby closing the wound.

It should be understood that various aspects disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques).

What is claimed is:

1. A tissue anchor used in repairing a wound after an endoscopic resection procedure, the tissue anchor comprising:
an elongated main body defining a channel therethrough, the main body having a proximal end portion, and a distal end portion configured to puncture tissue; and
at least two tines coupled to the main body, wherein the at least two tines are configured to move relative to the main between a stowed configuration, and a deployed configuration, in which the at least two tines flare outwardly relative to the main body.

2. The tissue anchor according to claim 1, wherein the at least two tines are linear in the stowed configuration.

3. The tissue anchor according to claim 1, wherein the at least two tines are arcuate in the deployed configuration.

4. The tissue anchor according to claim 1, wherein the at least two tines are fabricated from a shape memory material configured to transition the at least two tines from the stowed configuration to the deployed configuration when the at least two tines reach a preset, threshold temperature.

5. The tissue anchor according to claim 1, wherein the at least two tines have an outer surface that is circumferentially-aligned with or positioned radially inward from an outer surface of the main body when the at least two tines are in the stowed configuration.

6. The tissue anchor according to claim 1, wherein each of the at least two tines has a first end attached to the distal end portion of the main body, and a free second end.

7. The tissue anchor according to claim 6, wherein the free second end is perpendicular to a longitudinal axis defined by the main body when the at least two tines are in the deployed configuration.

8. The tissue anchor according to claim 1, wherein the at least two tines are positioned on the main body in opposition to one another.

9. The tissue anchor according to claim 1, wherein the at least two tines include four tines positioned circumferentially spaced from one another about the main body.

10. The tissue anchor according to claim 1, wherein the main body defines at least two elongated cutouts therein, the at least two tines being positioned within the respective at least two elongated cutouts when the at least two tines are in the stowed configuration.

11. The tissue anchor according to claim 1, wherein the main body has a tubular shape, and the channel is cylindrical.

12. The tissue anchor according to claim 1, wherein the channel is opened at each of the proximal and distal end portions of the main body.

13. The tissue anchor according to claim 1, wherein the distal end portion of the main body has at least one tooth extending distally therefrom, the at least one tooth being configured to puncture the tissue during a distal insertion of the main body into the tissue.

14. The tissue anchor according to claim 13, wherein the distal end portion of the main body has a distal-most edge having a first portion having a concave shape, and an opposite second portion having a concave shape, the at least one tooth being two teeth extending distally from the respective first and second portions of the distal-most edge.

15. The tissue anchor according to claim 1, wherein when the at least two tines are in the deployed configuration, the at least two tines have a convex, proximal-facing surface and a concave, distal-facing surface.

16. The tissue anchor according to claim 1, wherein the at least two tines and the main body are monolithically formed with one another.

17. A tissue anchor used in repairing a wound after an endoscopic resection procedure, the tissue anchor comprising:
an elongated main body including a proximal end portion, and a distal end portion, the main body defining a channel that extends from the proximal end portion to the distal end portion;
at least one tooth extending distally from the distal end portion of the main body, the at least one tooth being configured to puncture tissue during a distal insertion of the main body into the tissue; and
at least two tines having a first end coupled to the main body, and a free second end, wherein the at least two tines are configured to transition between a stowed configuration, and a deployed configuration, the free second end of the at least two tines are positioned a greater radial distance from an outer surface of the main body when the at least two tines are in the deployed configuration than when the at least two tines are in the stowed configuration.

18. The tissue anchor according to claim 17, wherein the at least two tines are linear in the stowed configuration, and arcuate in the deployed configuration.

19. The tissue anchor according to claim 17, wherein the at least two tines are fabricated from a shape memory material configured to transition the at least two tines from the stowed configuration to the deployed configuration when the at least two tines reach a preset, threshold temperature.

20. The tissue anchor according to claim 17, wherein the main body defines at least two elongated cutouts therein, the at least two tines being positioned within the respective at least two elongated cutouts when the at least two tines are in the stowed configuration.
